(19) United States
(12) Patent Application Publication
(10) Pub. No.: US 2010/0093862 A1
(43) Pub. Date: Apr. 15, 2010

(54) POTENTIATION OF CELLULAR IMMUNITY USING HISTONE DEACETYLASE (HDAC) INHIBITORS.

(75) Inventors: Toshihiko Torigoe, Hokkaido (JP); Noriyuki Sato, Hokkaido (JP); Hiroko Asanuma, Hokkaido (JP); Nobuhiro Fujii, Hokkaido (JP); Masaho Ishino, Hokkaido (JP)

Correspondence Address: QUINN EMANUEL;KODA & ANDROLIA, 865 S. FIGUEROA STREET, 10TH FLOOR, LOS ANGELES, CA, 90017, US

(73) Assignee: Sapporo Medical Univeristy and Japan Science and Technology Agency

(21) Appl. No.: 12/448,073

(22) PCT Filed: Dec. 6, 2007
(86) PCT No.: PCT/JP2007/074067

§ 371 Date: Jun. 5, 2009

Related U.S. Application Data
(60) Provisional application No. 60/873,223, filed on Dec. 6, 2006.

(51) Int. Cl. A61K 31/19; C07C 53/126; C12Q 1/68; C12Q 1/02; A61P 37/04; A61P 31/00; A61P 35/00

(52) U.S. Cl. 514/557; 562/512; 435/6; 435/29

(57) ABSTRACT

[Technical Problem] The present invention provides an enhancer of cellular immunity, and provides applications to carcinogenesis or preventive therapy against cancer recurrence, immunotherapy for cancer, treatment for pathogenic microorganism infection, and the like.

[Technical Solution] The present invention comprises the substance having an inhibitory activity of histone deacetylase (HDAC) as an active component.

### TECHNICAL FIELD

The present invention relates to a method for enhancing cellular immunocompetence by increasing the level of expression of major histocompatibility complex (MHC) class I antigen on a cell surface by using histone deacetylase (HDAC) inhibitors. Namely, the present invention relates to a method for enhancing cellular immunity using HDAC inhibitors. Moreover, the present invention relates to an application of such methods for enhancing cellular immunity to a prevention of carcinogenesis or cancer recurrence, an immunotherapy of an existing cancer, or a treatment of infection by pathogenic microorganism.

### BACKGROUND ART

Biological immunity is generally classified into humoral immunity depending on antibody production and cellular immunity depending on targeted cellular injury by lymphocytes. The most important immunity in resisting against pathogenic microorganism infected in cells or a cellular disease such as a cancer is cellular immunity. Therefore, enhancing such cellular immunocompetence is expected to exert efficacy in wide areas such as a prevention or treatment of cancer and a treatment of infection by pathogenic microorganism such as virus and bacteria.

In cellular immunity, lymphocytes recognize target cells such as cancer cells and virus-infected cells, and in these mechanisms the MHC class I antigen molecule is playing important roles. Human MHC is called human leukocyte antigen (HLA). For example, antigen peptides as degradation products from cancer antigen proteins in cancer cells or antigen peptides as degradation products from virulence factor-derived proteins in cells infected with virus and the like, each bind to the HLA class I molecule and appear on a cell surface. Among immunocompetent cells, T cells recognize the antigen peptide/HLA complex on a target cell surface by the T-cell antigen receptor on the cell surface and discriminate between normal cells and cancer/infected cells. This means that, when the expression of the HLA class I molecule is suppressed, the target cell-recognizing mechanism by the T cell would not function normally.

In the case of humans, the MHC class I antigen is composed of a hetero-dimer of two molecules, that is a heavy chain encoding mainly three kinds of genes: HLA-A, HLA-B and HLA-C, and a light chain encoding one gene called β2-microglobulin (B2M). When B2M is not normally expressed, the formation of a mature HLA complex and the transportation to the cell membrane surface are impaired, resulting in lowered expression of the products of all the HLA-A, HLA-B, and HLA-C genetic loci.

MHC class I antigen is expressed on the cellular surface of almost all somatic cells excluding testicular cells. However, in certain cancer cells such as breast and prostatic cancer cells, the expression of cell surface MHC class I antigen is frequently decreased. As to the mechanism of the decrease of the expression of MHC class I antigen, a downregulation mechanism due to the gene mutation in the heavy or light chain is known (Non-patent document 1), but its details are unknown. In various viral infections, on the other hand, inhibition of the expression of class I antigen has also been known, and its various mechanisms have been reported. Interestingly, it has been known that each virus has plural intricate strategies to inhibit the class I expression. For example, cytomegalovirus expresses four genes (US3, US2, US11 and US6) which interfere with the MHC class I pathway: US3 inhibits the export of the MHC class I complexes from the endoplasmic reticulum, US2 and US11 induce the rapid destruction of the MHC class I complexes by the proteasome, and US6 inhibits the peptide supply into the endoplasmic reticulum, all interfering with the function of MHC class I (Non-patent document 2). HIV virus not only inhibits the transcription of B2M and class I genes through Tat (Non-patent document 3) but also interferes with the MHC class I function by altering the transport pathway for the class I product through Nef (Non-patent document 4).

In this way, cancer tissues and virus-infected cells evade immunological surveillance by adroitly inhibiting the expression of HLA class I antigen molecule. Therefore, if the expression of class I molecule is increased in these tissues and cells, the cellular immunocompetence of host would be expected to rise, providing a marked efficacy toward prevention and treatment of cancers and infections.

On the other hand, epigenetic regulation without change in the base sequence of genes has recently been known to be extensively involved in carcinogenesis and the like. Histone acetylation, which regulates the chromatin activity, is considered one of factors playing important roles in epigenetic regulation, and histone deacetylase (HDAC) that regulates its level is attracting attention as a molecular target for cancer and the like (Non-patent document 5). Various HDAC inhibitors suppressing the action of HDAC have been known (Non-patent documents 5 and 6). The specific selectivity of these HDAC inhibitors for HDAC belonging to each class is not necessarily strict, but for example, it has been reported that tricostatin A (TSA) inhibits class I and II HDAC while valproic acid (VPA) is preferably inhibits class I MAC compared with class II HDAC, and the like (Non-patent documents 5 and 6).
Non Patent Citation 1: Seliger, B., et al., Semin Cancer Biol 2002, 12: 3-13.
Non Patent Citation 2: Mirandola, P., et al., J Infect Dis. 2006 Apr. 1; 193(7): 917-26.
Non Patent Citation 3: Carroll, I R., et al., Mol Immunol. 1998 December; 35(18): 1171-8.
Non Patent Citation 4: Lubben, N B., et al., Mol Biol Cell. 2007.
Non Patent Citation 5: Kim, D H., et al., J Biochem Mol Biol. 2003. Jan. 31; 36(1): 110-9.
Non Patent Citation 6: Gottlicher, M., et al., EMBO J. 2001. Dec. 17; 20(24):6969-78.

### DISCLOSURE OF INVENTION

### Technical Problem

In cancer cells and pathogenic microbe-infected cells, the expression of the MHC class I antigen molecule is frequently decreased. Therefore, virus-infected cells and cancer cells escape from the immunological surveillance of host, and this is considered to be a cause of inability of eliminating viruses and cancers by the immunocompetence in a living body. In order to prevent and treat an infection, a cancer and the like by the immunocompetence, it is important to enhance the expression level of MHC class I antigen on a cellular surface which is decreased in pathogenic microbe-infected cells or cancer cells, and hence a method to solve this problem had been sought.

The present inventors found that, in many cancer cells, the expression level of MHC class Ito the cellular surface is decreased despite the absence of their mutational change in both MHC class I heavy chain and light chain genes. They further found that, in certain cancer cells, the cause of the suppression of the expression of MHC class I antigen is ascribable to deacetylation of histone bound to the B2M gene. Then, it was demonstrated that HDAC inhibitors, when added to the cells whose expression of B2M was decreased, increased the expression level of MHC class I on a cellular surface as well as the level of B2M protein. In other words, it has been proved that, in cells where the suppression of the expression of MHC class I antigen is induced, the use of HDAC inhibitors elevated acetylation of the histone bound to the B2M gene and increased the level of B2M protein, resulting in increased expression levels of MHC class I antigen on a cellular surface. On the other hand, as to the decreased expression levels of MHC class I in virus infection, involvement of various and intricate systems has been known as described above. Surprisingly, as a result of the intensive studies of the present inventors, the use of deacetylation inhibitors of histone has demonstrated to bring about uniformly the increase of the expression levels of MHC class I antigen in various virus infection systems, and this contributed to the accomplishment of the present invention.

### Technical Solution

Namely, the enhancer of cellular immunity according to the present invention is characterized in that a substance having an inhibitory activity of histone deacetylase (HDAC) is comprised as an active ingredient.

Further, in the enhancer of cellular immunity according to the present invention, a substance having an inhibitory activity of HDAC is preferably valproic acid.

Also, the enhancer of cellular immunity according to the present invention, is preferably used for treatment or prevention of a disease in which β2-microglobulin (B2M) expression is suppressed at the disease site.

Alternatively, the enhancer of cellular immunity according to the present invention, is preferably used for treatment or prevention a disease in a subject suffering from, or at risk of, an infection caused by a cell-invading pathogenic microorganism/factor; or in a subject suffering from, or at risk of, a disease. The pathogenic microorganism is preferably a cell-invading bacteria. Also, the factor is preferably a virus. Further, the virus is more preferably one or plurality of viruses selected from the group consisting of members of the herpesvirus, papovavirus, lentivirus, and paramyxovirus families. The disease is preferably a cancer, more preferably prostate cancer or oral cancer, and furthermore preferably breast cancer.

Next, the enhancer of HLA class I expression according to the present invention is characterized by comprising a substance having an inhibitory activity of HDAC as an active ingredient.

Also, in the enhancer of HLA class I expression according to the present invention, the substance having an inhibitory activity of HDAC is preferably valproic acid.

Then, the enhancer of B2M expression according to the present invention is characterized by comprising a substance having an inhibitory activity of HDAC as an active ingredient.

Also, in the enhancer of B2M expression according to the present invention, the substance having an inhibitory activity of HDAC is preferably valproic acid.

Then, the method for enhancing cellular immunity according to the present invention is characterized by comprising a step dosing a subject a composition containing a substance having an inhibitory activity of HDAC as an active ingredient.

Also, in the method for enhancing cellular immunity according to the present invention, the substance having an inhibitory activity of HDAC is preferably valproic acid.

Also, the method for enhancing cellular immunity according to the present invention is preferably used for treatment or prevention of a disease in which β2-microglobulin(B2M) expression is suppressed at the disease site.

Alternatively, in the method for enhancing cellular immunity according to the present invention, the subject is preferably infected with a pathogenic microorganism, and the pathogenic microorganism is more preferably a virus, and the virus is furthermore preferably one or plurality of viruses selected from the group consisting of members of the herpesvirus, papovavirus, lentivirus, and paramyxovirus families. Also, in the present invention, the disease is preferably a cancer, and the cancer is more preferably prostate cancer or oral cancer, and the cancer is furthermore preferably breast cancer.

Namely, the method for enhancing cellular immunity according to the present invention can be used for prevention or treatment of a disease in a subject suffering from, or at risk of, an infection caused by a cell-invading pathogenic microorganism/factor comprising any virus; or in a subject suffering from, or at risk of, cancer such as breast cancer, prostate cancer, oral cancer, or the like.

Then, the method for screening subjects whose cellular immunity is to be enhanced by using a substance having an inhibitory activity of HDAC according to the present invention is characterized by comprising a step obtaining an infective tissue or cancer tissue from the subjects, a step extracting B2M or mRNA encoding B2M from said tissue, and a step comparing the amount of said B2M or said mRNA encoding B2M with an amount of a control.

Moreover, the method for screening subjects whose cellular immunity is to be enhanced by using a substance having an inhibitory activity of HDAC according to the present invention is characterized by comprising a step obtaining an infective tissue or cancer tissue from the subject, a step performing chromatin immunoprecipitation with said tissue to obtain a precipitate, and performing PCR amplification of DNA fragments of B2M from said precipitate to obtain amplified DNA fragments, and a step determining whether said amplified DNA fragments exist.

Also, in the screening method according to the present invention, the substance having an inhibitory activity of HDAC is preferably valproic acid.

### ADVANTAGEOUS INVENTION

By the enhancer of cellular immunity, the enhancer of HLA class I expression, the enhancer of B2M expression, or the method for enhancing cellular immunity according to the present invention, an expression level of MHC class I antigen can be increased in target cells. As a result, the injurious effect on the target cell by the cytotoxic T cell is enhanced in a host. Therefore, by using the method of the invention, direct treatment can be performed or the therapeutic effect of a concurrent therapy can be increased in a disease such as a microbial infection, a cancer and the like.

Also, by the method for screening subjects whose cellular immunity is to be enhanced by using a substance having an inhibitory activity of HDAC according to the present invention, target cells whose expression is decreased through the decreased protein level of HLA class I light chain (B2M) can be selected, and hence a subject suffering from, or at risk of occurrence/recurrence of, a disease in which B2M expression is suppressed at the diseased site can be screened. Similarly, treatment of a disease such as a microbial infection, a cancer and the like, can be performed and the therapeutic effect of a concurrent therapy can be increased.

### BRIEF DESCRIPTION OF THE DRAWINGS

FIG. 1 is a drawing showing that intracellular B2M gene-binding histone proteins are deacetylated by chromatin sedimentation. The left panel is an example in which MCF-7 human breast cancer cells were cultured in the presence of TSA, and the right panel is an example in which HMC-1 human breast cancer cell line was cultured in the presence of VPA. Since in both cases, B2M gene was almost undetected in anti-acetylated histone antibody immunoprecipitates in untreated state, it is found that histone proteins were deacetylated in B2M gene region. B2M genes have been detected in the same immunoprecipitates by acting TSA or VPA.

FIG. 2 is a drawing showing that the level of intracellular B2M protein is increased by HDAC inhibitor. The left panel is an example in which HMC-1 human breast cancer cells were cultured and the right panel is an example in which MCF-7 human breast cancer cell line was cultured, for 24 hours in the presence of TSA, respectively. In both cells, it was shown that B2M protein which was in extremely lower level in non-treated state was remarkably increased by the action of an HDAC inhibitor TSA. In contrast, there is no great change in the levels of HLA class I heavy chain and actin protein which are expressed in higher level even in non-treated state.

FIG. 3 is a drawing showing that the level of MHC class I antigen on a cell surface was increased by an HDAC inhibitor TSA. The left panel is the example in which HMC-1 human breast cancer cells were cultured and the right panel is an example in which MCF-7 human breast cancer cell line was cultured, for 24 hours in the presence of TSA, respectively. In both cells, it was shown that MHC class I antigen on a cell surface which was in extremely lower level in non-treated state was increased by about ten times by the action of an HDAC inhibitor TSA. The longitudinal axis indicates cell count, and the horizontal axis indicates fluorescence intensity.

FIG. 4 is a drawing showing that the level of MHC class I antigen on a cell surface was increased by an HDAC inhibitor VPA, the safety of which has been established in human. HMC-1 human breast cancer cells were cultured for 48 hours in the presence of 4 mM VPA. It is shown that MHC class I antigen on a cell surface which is in lower level in non-treated state is increased by about ten times by the action of an HDAC inhibitor VPA. The longitudinal axis indicates cell count, and the horizontal axis indicates fluorescence intensity.

FIG. 5 is a drawing showing a cancer case in which the expression of HLA class I antigen (heavy chain and light chain B2M protein) on a cell surface is positive (upper two panels, Case A), a cancer case of negative (lower two panels, Case B). The left upper and lower two panels are the drawing in which the breast cancer samples were immunostained with anti-HLA class I heavy chain antibody (EMR8-5 antibody), and the right upper and lower two panels are the drawing in which the same breast cancer samples were immunostained with anti-B2M antibody (EMRB6 antibody). The drawings show that there are HLA class I positive cancer (Case A) and negative cancer (Case B) in human breast cancer tissues.

FIG. 6 Immunostaining was performed with similar anti-HLA class I heavy chain antibody (EMR8-5 antibody) to that in FIG. 5, and cell surface expression of HLA class I antigen was searched on various types of human cancer tissues. As shown in the table, it is indicated that the frequencies of the expressions are different depending on the types of cancer, but there exist the cancer in which HLA class I antigen is positive and the cancer of negative or of decreased expression with regard to all the cancer types. It is noticed that the frequency in which the expression of HLA class I antigen is decreased is higher especially in breast cancer and prostate cancer.

FIG. 7 is a drawing showing that the expression level of HLA class I antigen is increased in tumor of VPA-administrated mouse (VPA (+)) compared to control group (VPA (−)).

FIG. 8 is a drawing showing that the expression level of B2M is increased in tumor of VPA-administrated mouse (VPA (+)) compared to control group (VPA (−)).

FIG. 9 is a drawing showing that the expression level of HLA class I antigen is increased in human immunodeficiency virus (HIV) persistent infection T cells in the presence of VPA (VPA (+)) compared to control group (VPA (−)).

FIG. 10 is a drawing showing that the expression level of HLA class I antigen is increased in papilloma virus persistent infection epithelial cell line (HeLa cells) in the presence of VPA (VPA (+)) compared to control group (VPA (−)).

FIG. 11 is a drawing showing that the expression level of HLA class I antigen is increased in EB virus persistent infection FIG. 11 B cells (LG2EBV cells) in the presence of VPA (VPA (+)) compared to control group (VPA (−)).

FIG. 12 is a drawing showing that the expression level of HLA class I antigen is increased in mumps virus persistent infection B cells (Akata-MP1 cells) in the presence of VPA (VPA (+)) compared to control group (VPA (−)).

FIG. 13 is a drawing showing that the expression level of HLA class I antigen is increased in measles virus persistent infection B cells (Raji-ZH cells) in the presence of VPA (VPA (+)) compared to control group (VPA (−)).

### BEST MODE FOR CARRYING OUT THE INVENTION

Hereafter, preferred embodiments of the present invention are explained. The first embodiment of the present invention relates to a composition containing a substance having an inhibitory activity of HDAC as an active component.

“A substance having an inhibitory activity of HDAC” may be anything as long as it is the HDAC inhibitor capable of being administered to human or animals. For example, one of the agents having HDAC inhibitory action, the safety of which is most established in human at this time is valproic acid (VPA). In the case of VPA, although it is possible to administer orally to human at a daily dosage of about 1000 mg to 2400 mg, the dosage can be adjusted accordingly depending on body weight and age, pharmacokinetics, safety, effect, purpose, administration method and the like.

As a substance having an inhibitory activity of HDAC, any HDAC inhibitor besides VPA can be used equivalently. For example, at the time period of the present application, the agents such as phenylbutyric acid, FK228 (Depsipeptide), SAHA (suberoylanilide hydroxamic acid), PXD101, CI-994 (N-acetyl dinaline), MGCD0103, Pivanex, CRA-024781, MS-275, LBH589, MG989, LAQ-824, and NVP-LAQ824 are already under clinical trials in another use separating from the present invention. In addition, numerous HDAC inhibitors such as TSA, Trapoxin, CHAP, Apicidin and Depudecin are known. Therefore, as “a substance having an inhibitory activity of HDAC” it is possible to use an HDAC inhibitor having equivalent action and/or specificity to VPA in the effect of the present invention. Such HDAC inhibitor can be referred to as an inhibitor to the HDAC of class I and/or class II.

Also, a composition containing the substance having an inhibitory activity of HDAC in the present invention as an active ingredient is used for treatment or prevention of the diseases in which B2M expression is suppressed at the diseased site. The aforementioned diseased site means, for example if it is cancer, cancer cells; if it is infection, the cells infected by pathogenic microorganism.

Furthermore, although a subject to be administrated with the composition containing the substance having an inhibitory activity of HDAC in the present invention as an active ingredient is not limited particularly, the subject includes a cancer patient, a subject at a risk of onset or recurrence of cancer, or a subject infected with pathogenic microorganism such as virus and the others, or a subject at a risk of the infection.

Infection with pathogenic microorganism in the present invention is not particularly limited, and it includes, for example, the infections by viruses containing viruses of the herpesvirus family including EB virus, cytomegalovirus, HSV, HHV-7 and the like, viruses of the papovavirus family including papillomavirus and the like, viruses of the parvovirus family, viruses of the adenovirus family, viruses of the hepadnavirus family including HBV and the like, viruses of the poxvirus family, viruses of the picornavirus family including coxsackievirus belonging to enterovirus, HAV and the like, viruses of the coronavirus family including SARS virus, viruses of the togavirus family, viruses of the flavivirus family including HCV, West Nile fever virus and the like, viruses of the rhabdovirus family, viruses of the filovirus family, viruses of the paramyxovirus family including measles virus, epidemic parotitis virus (mumps virus), parainfluenza virus and the like, viruses of the arenavirus family, viruses of the bunyavirus family including Hantaan virus and the like, viruses of the orthomyxovirus family including influenza virus, viruses of the retrovirus family including HIV-1/2 of the lentivirus subfamily, FIV and HTLV-1/2 of the oncovirus subfamily and the like, viruses of the reovirus family including rotavirus, and the other various viruses, as well as the infections by bacteria, mycoplasmas, rickettsias, fungi, parasites such as protozoans, and the like, and also any infections of which virulence factors can not be necessarily specified. In other words, the aforementioned infections are not particularly limited, but it is possible to apply to the subject having or at a risk of, for example, reovirus infection, SARS, retrovirus infection, yellow fever, Japanese encephalitis, dengue fever, West Nile fever, bovine viral diarrhea-mucosal disease, hog cholera, border disease, hepatitis C, foot-and-mouth disease, poliomyelitis (acute poliomyelitis), hepatitis A, cold, rubella, Sindbis virus infection, equine viral arteritis, simian hemorrhagic fever, norovirus infection, sapovirus infection, rabbit hemorrhagic disease, feline calicivirus disease, vesicular exanthema of swine, hepatitis E, Marburg disease, Ebola hemorrhagic fever, rabies, vesicular stomatitis, measles, peste des petits ruminants, canine distemper, rinderpest, epidemic parotitis, Menangle virus infection, Newcastle Disease, RS virus infection, human metapneumovirus infection, Nipah virus infection, influenza, Akabane disease, Hantavirus pulmonary syndrome, hemorrhagic fever with renal syndrome, lymphocytic choriomeningitis, Lassa fever, Argentine hemorrhagic fever, Bolivian hemorrhagic fever, AIDS, adult T-cell leukemia, or varicella, smallpox, herpes zoster, cytomegalovirus infection, adenovirus infection, hepatitis B, and other diseases.

In addition, a composition containing the substance having an inhibitory activity of HDAC in the present invention as an active ingredient can be used alone or as an adjuvant in treatment or prevention of the infections with pathogenic microorganisms to supplement the treatment and preventive action for the other infections known to those skilled in the art.

Also, a composition containing the substance having an inhibitory activity of HDAC in the present invention as an active ingredient can be used as an anti-cancer agent and/or as an adjuvant in treatment or prevention of cancers to supplement the other anti-cancer treatment. The most preferred embodiment in the prevention of the cancer is a prevention of cancer recurrence.

Although the cancers which are the subject of treatment or prevention in the present invention is not particularly limited, various cancers such as lung cancer, renal cell cancer, hepatocellular cancer, colon cancer and bladder cancer can be listed, preferably prostate cancer or oral cancer, more preferably breast cancer. On the other hand, the enhancer of cellular immunity of the present invention can be preferably applied in individual cases as far as B2M expression is suppressed, not necessarily in the diseases in which B2M suppression is observed frequently (for example, breast cancer and prostate cancer).

Also, methods of the anti-cancer treatment are not particularly limited, but they includes various treatments such as radiotherapy, thermotherapy, chemotherapy (for example, anti-cancer agents such as anastrozole, cyclophosphamide, irinotecan, cytarabine, paclitaxel, docetaxel, busulfan, carboquone, mitobronitol, dacarbazine, melphalan, procarbazine, doxifluridine, fluorouracil, camofur, mercaptopurine, methotrexate, cytarabine ocfosphate, tegafur, carboplatin, cisplatin, thiotepa, doxorubicin, epirubicin, aclarubicin, L-asparaginase, mitomycin C, medroxyprogesterone, Krestin, tamoxifen, toremifene, vinorelbine and etoposide, or those using pharmaceutically accepted salts thereof, and the like), gene therapy (those introduced with tumor suppressor gene such as p53, those using tumor cells introduced with immunostimulatory cytokine, and the like), surgical therapy and alimentary therapy, and preferably immunotherapy. Cancer vaccine therapy using tumor antigen peptide, DNA vaccines that introduce peptide antigen or tumor-cell gene through vectors/carriers such as viruses into living bodies, RNA vaccines utilizing RNA in tumor cells, the treatments using cytokine and dendritic cells as antigen-presenting cells in combination, monoclonal antibody therapy, and the like are included in the immunotherapy.

In addition, a composition containing the substance having an inhibitory activity of HDAC in the present invention as an active component can be also used for producing the pharmaceuticals for the use to enhance cellular immunity. Such enhancers of cellular immunity are used for prevention, recurrence prevention and the like, besides treatment as well as the improvement of disease status, relating to various diseases according to the present invention.

Also, a method of administering a composition containing the substance having an inhibitory activity of HDAC in the present invention as an active component is not particularly limited, but, it is possible to administrate to a subject orally or parenterally (for example, intravenously including intravenous infusion, arterially, subcutaneously, intramuscularly, intraperitoneally, topically or the like). The subject for administration may be any animals, for example, lizard, chicken, domestic duck, wild duck, mouse, rat, canine, feline, sheep, goat, bovine, equine, porcine, monkey, human and others can be considered for them, and preferably mammals including human.

Then, a composition containing the substance having an inhibitory activity of HDAC in the present invention as an active component can be prepared by formulating together with pharmaceutical carriers which are used usually. For example, HDAC inhibitor which is an active component can be prepared for oral administration as liquid oral formulation such as syrup, or processed into extract, powder and the like and blended with pharmaceutically acceptable carriers to prepare oral formulation such as tablet, capsule, syrup, emulsion, granule and powder.

As pharmaceutically acceptable carriers, various substances which are usually used are employed, and for example, it is possible to blend excipient, lubricant, binder, disintegrator in a solid formulation, or solvent, excipient, solubilizing agent, solubilizer, emulsifier, suspending agent, binder in a liquid formulation, and the like. In addition, it is also possible to add formulation additives such as preservative, antioxidant, colorant and sweetener as needed.

A composition containing the substance having an inhibitory activity of HDAC as an active component in the present invention can be used alone, but since it is used for the purpose of the enhancement of cellular immunity, it can be preferably used in combination with antigen-specific vaccines such as cancer antigen-specific vaccines and viral vaccines, as well as other antigen-nonspecificimmunostimulants, for example, cytokines such as interferone and various adjuvants. The pharmaceutical agents which are used in combination can be used either by mixing or separately with the pharmaceutical agents containing an HDAC inhibitor in the present invention.

Next, the second embodiment of the present invention relates to a method for enhancing cellular immunity including a step of administrating a composition containing the substance having an inhibitory activity of HDAC as an active component to a subject. That is, it is a method for enhancing cellular immunity in a subject suffering from, or at a risk of, a disease derived from decreased immunity, wherein the method includes a step of administrating a composition containing pharmaceutically acceptable carriers and the substance having an inhibitory activity of HDAC to the subject in a dosage of enhancing cellular immunity.

A method for screening a subject having the disease in which B2M expression is suppressed at the diseased site, adequate for the method for enhancing cellular immunity in the present invention, or accordingly, a subject having or at a risk of occurrence/reoccurrence of the disease in which B2M expression is suppressed at the disease site, adequate for the method for enhancing cellular immunity according the present invention is also within the scope of the present invention, and the third embodiment of the present invention. The followings are included in such a screening:

1) a step of obtaining a diseased site such as cancer tissue from a subject;

2) a step of extracting B2M or mRNA(s) encoding B2M from the above tissue; and

3) a step of comparing an amount of the B2M or the mRNA(s) encoding B2M with an amount of a control.

Also, tissue can be obtained through lavage fluid, abrasion collection, body fluids such as blood, spinal fluid, lymph, urine, saliva, ascitic fluid, pleural fluid and exudate, or the like besides through biopsy which is usually used for each organ.

Then, comparison of the amounts of B2M or mRNA encoding B2M can be achieved by using a method known to those skilled in the art, for example by using mass spectrometer or an immunological method such as ELISA, radioimmunoassay, fluorescent antibody method, western blotting, immunohistochemical staining or the like; a genetic method such as northern blotting, RT-PCR, microarray or the like; or others.

The screening is adequate for primary screening through which a subject to be applied the method of the present invention is selected, however, more stringently, it is preferred to further perform the screening including the following steps or to further narrow the subject to be selected by performing them from the first.

1) a step of obtaining a diseased site such as cancer tissue from a subject;

2) the step of performing chromatin immunoprecipitation with the above tissue to obtain a precipitate, and performing PCR amplification of a DNA fragment of B2M from the precipitate; and

3) the step of evaluating the presence or absence of the amplified DNA fragment.

There is a possibility of suppression of the expression of B2M gene by the presence of HDAC activity in a subject in which the DNA fragment of B2M is not amplified by this procedure, and the effect by application of the treatment method of the present invention and the like is expected. Herein, “chromatin immunoprecipitation” is a method of affinity-precipitation of the DNA associating with chromatin together with the chromatin, any appropriate methods can be used, however, for example, it is possible to maintain the associating status between chromatin and the DNA by pre-treating tissue cells with a cross-linker such as 1% formaldehyde (for example for 10 minutes at room temperature) followed by precipitating chromatin and the associated DNA fragment after lysing the cells using RIPA buffer which are used usually, and the like, and shearing the DNA using an ultrasonic wave homogenizer and the like (see Assam El-Osta et al., (2002) MOLECULAR AND CELLULAR BIOLOGY 22(6) p 1844-1857).

In addition, the method for enhancing cellular immunity which is the second embodiment of the present invention is a method for enhancing HLA class I expression in a subject including a step of administrating the composition containing the substance having an inhibitory activity of HDAC as an active component to the subject. Such composition is used for treatment or as a preventive agent of the infection by pathogenic microorganism such as virus or used as an anti-cancer agent in treatment of the disease in which B2M is suppressed, for example, in treatment of cancer or used for the assistance of other treatment methods of the disease, but not limited to.

Also, a method for enhancing cellular immunity according to the present invention is a method for enhancing B2M expression in a subject including a step of administrating a composition containing the substance having an inhibitory activity of HDAC as an active component to the subject. Such composition is used for treating or preventing cancer, for example, alone or in combination with other treatment methods, but not limited to.

Unless otherwise specified, the terms used herein have the meanings which are normally understood and used by those skilled in the art. Also, unless otherwise specified, the methods and techniques used herein can be executed according to the common use of procedures well known in the technical art. References being described these procedures and the like are listed as for example Sambrook et al., Molecular Cloning: A Laboratory Manual, Cold Spring Harbor Laboratory Press (1989 and 2001); Ausubel et al., Current Protocols in Molecular Biology, Greene Publishing Associates (1992 and supplement); and Harlow and Lane, Using Antibodies: A Laboratory Manual, Cold Spring Harbor Laboratory Press (1999) and the like and the instruction manual provided by providers described in the present description, and the like. Cited documents such as scientific literatures, patents, patent applications cited herein are incorporated by reference in their entirely.

### EXAMPLE

Hereafter, the present invention is illustrated in Examples, but the scope of the present invention is in no way restricted by the Examples.

### Mode for the Invention 1

The present example shows that the histone proteins binding to the intracellular B2M gene region are deacetylated and this deacetylation is cancelled by HDAC inhibitor. The following shows the procedure.

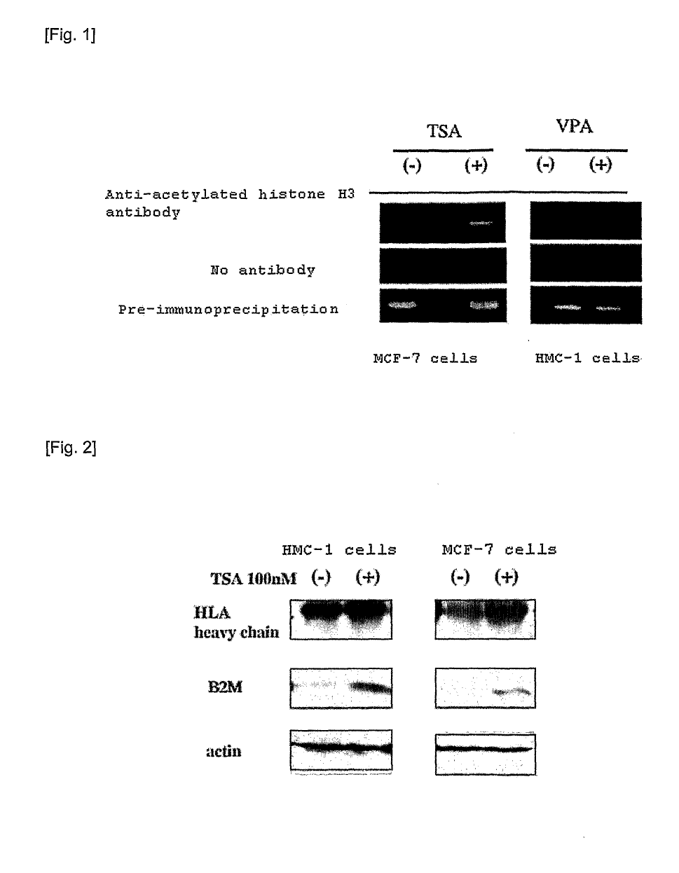

Human breast cancer cell lines, that is, MCF-7 cells and HMC-1 cells were used to culture in the presence of 100 nM TSA (Sigma, USA) for 24 hours or 4 mM VPA (Sigma, USA) for 48 hours, respectively followed by extracting chromatin from the cells according to the manufacturer's instructions (Upstate Inc., USA) and immunoprecipitating the chromatin using anti-acetylated histone H3 antibody (Upstate Inc., USA) (see Assam El-Osta, et al. 2002. MOLECULAR AND CELLULAR BIOLOGY 22(6). p. 1844-1857). The genes binding to the acetylated histones were amplified by using PCR with the B2M gene-specific primers (GAAAACGGGAAAGTCCCTCT and AGATCCAGCCCTGGACTAGC), electrophoresed on 1% agarose gel followed by detecting with a transilluminator in the presence of ethidium bromide. The results showed that the histones of B2M gene region in any cancer cells were deacetylated. When histone acetylation by the action of HDAC inhibitors such as TSA and VPA was facilitated, B2M gene was detected together with acetylated histones (FIG. 1).

### Mode for the Invention 2

The present example shows that the expression of intracellular B2M protein is increased by HDAC inhibitor. The following shows the procedure.

(1) Using the same cells as those in the example 1, MCF-7 cells and HMC-1 cells were cultured in the presence of TSA, a HDAC inhibitor, (100 nM) for 24 hours respectively, followed by harvesting them.

(2) 1×106 cells are lysed in 100 ml cell lysate (RIPA buffer) and a soluble fraction is harvested as a lysate. SDS sample buffer was added to the lysate.

(3) The protein samples were loaded on 7.5% SDS polyacrylamide gel and electrophoresed.

(4) The proteins in the gel were transferred on a PVDF membrane (Millipore).

(5) The transferred membrane was immersed in 5% skim milk in PBS and blocked for about one hour.

(6) The transferred membrane was immersed in a primary antibody (anti-pan-HLA class I heavy chain antibody EMR8-5 (Hokudo, Sapporo), or anti-B2M antibody EMRB6 (Hokudo, Sapporo), or anti-actin antibody (DAKO), and incubated for one hour at room temperature.

(7) The transferred membrane was washed with a lavage PBS-T (0.05% Tween 20/PBS, pH7.4) three times.

(8) The transferred membrane was immersed in a secondary antibody peroxidase-labeled anti-mouse IgG antibody (KPL, USA) and incubated for one hour at room temperature.

(9) The transferred membrane was washed with a lavage PBS-T (0.05% Tween 20/PBS, pH7.4) three times.

(10) The transferred membrane was immersed in ECL kit (Amersham, USA) luminescent solution and subjected to color reaction for about one minute.

(11) Luminescent signals were detected by exposure to X ray film.

The results showed that although there was no great difference in the expression level of intracellular HLA heavy chain and actin proteins in the presence or absence of TSA, the level of B2M protein was remarkably increased by TSA treatment (FIG. 2).

### Mode for the Invention 3

Using the same cells as those in the examples 1 and 2, MCF-7 cells and HMC-1 cells were cultured for 24 hours in the presence of HDAC inhibitor TSA (100 nM) or for 48 hours in the presence of HDAC inhibitor VPA (4 mM), respectively, and the level of MHC class I antigen expressing on the cell surface was analyzed by a flow cytometer (Becton Dickinson). The following shows the procedure.

(1) 1×106 cells were floated in 100 ml PBS buffer, added a primary antibody (an antipan-HLA class I antibody W6/32 (Barnstable, C. J., Bodmer, W. J., Brown, G., Galfre, G., Milstein, C., Williams, A. F., and Zeigler, A. (1978) Cell 14, 9-20)) and incubated for 40 minutes at 4° C.
(2) The cells were washed twice with PBS.
(3) To the suspended cell was added a secondary antibody (FITC-labeled anti-mouse IgG antibody (IPL, USA)) and incubated for 40 minutes at 4° C.
(4) The cells were washed twice with PBS, floated in PBS added with 1% formalin followed by analyzing FITC level of the cell surface by a flow cytometer.

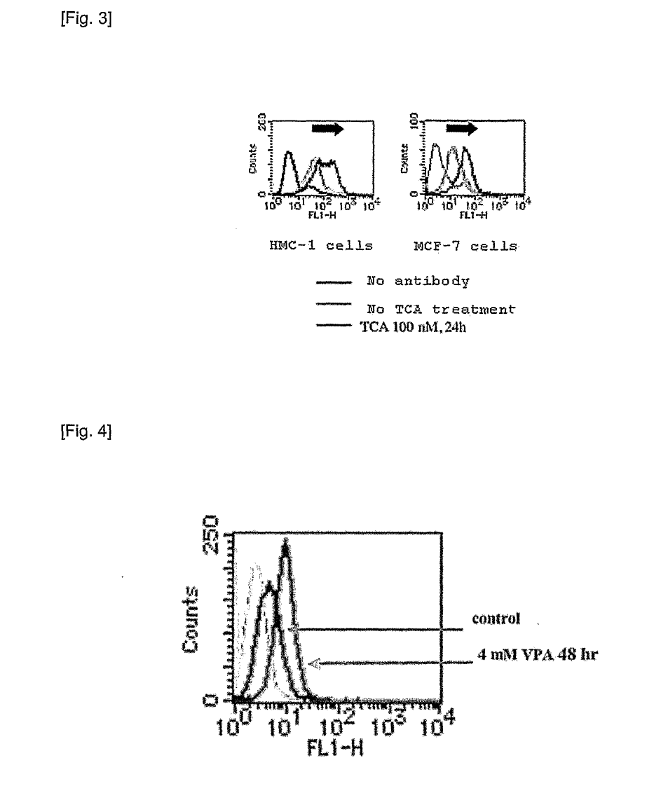

The result showed that MHC class I level of the cell surface in the presence of TSA or VPA was increased about 10 times (FIG. 3, FIG. 4).

### Mode for the Invention 4

In this example, immunohistochemical staining of human cancer tissues fixed with formalin was performed and the expression levels of MHC class I antigen in the actual human cancer tissues were analyzed. The following shows the procedure.

(1) The paraffin-embedded slices of human breast cancer tissue fixed with 20% formalin fixative were deparaffinized by ethyl alcohol.

(2) As a treatment for antigen activation, the slices were immersed in 0.01 mol/L citrate buffer (pH 6.0) and autoclaved (110° C. for 5 minutes).

(3) 0.5 ml of 5 mg/ml of anti-HLA class I heavy chain antibody (EMR8-5) or 0.5 ml of 5 mg/ml of anti-b2 microglobulin (B2M) antibody (EMRB6) as a primary antibody was added dropwise on the slices and incubated for one hour at room temperature.

(4) They were washed with a lavage PBS-T (0.05% Tween 20/PBS, pH7.4) three times.

(5) A secondary antibody peroxidase-labeled anti-mouse IgG antibody (Simple Stain MAX-PO, NICHIREI) was dropped on the slices and incubated for 30 minutes at room temperature.

(6) They were washed with a lavage PBS-T (0.05% Tween 20/PBS, pH7.4) three times.

(7) The slices were immersed in the mixed solution of hydrogen peroxide aqueous solution and DAB substrate (Simple Stain MAX-PO, NICHIREI), and developed for one to two minutes.

(8) The slices were washed with a running water for one minute.

(9) Hematoxylin nuclear stain was executed (one to two minutes). Hematoxylin nuclear stain was followed by the following procedure.

1. Take sections to water.
2. Place sections in Mayers Haematoxylin (filtrated) for 5 minutes.
3. Wash in tap water.
4. ‘Blue’ sections in tap water.
5. Place sections in 1% Acid Alcohol (70% Alcohol 99 ml plus conc. Hydrochloric Acid 1 ml) for a few seconds.
6. Wash in tap water.
7. Place sections in Eosin Solution (Eosin Yellowish 1.0 g in Distilled Water 100 ml) for 5 minutes.
8. Wash in tap water.
9. Dehydrate, clear.
10. Mount sections in Immumount or DPX.

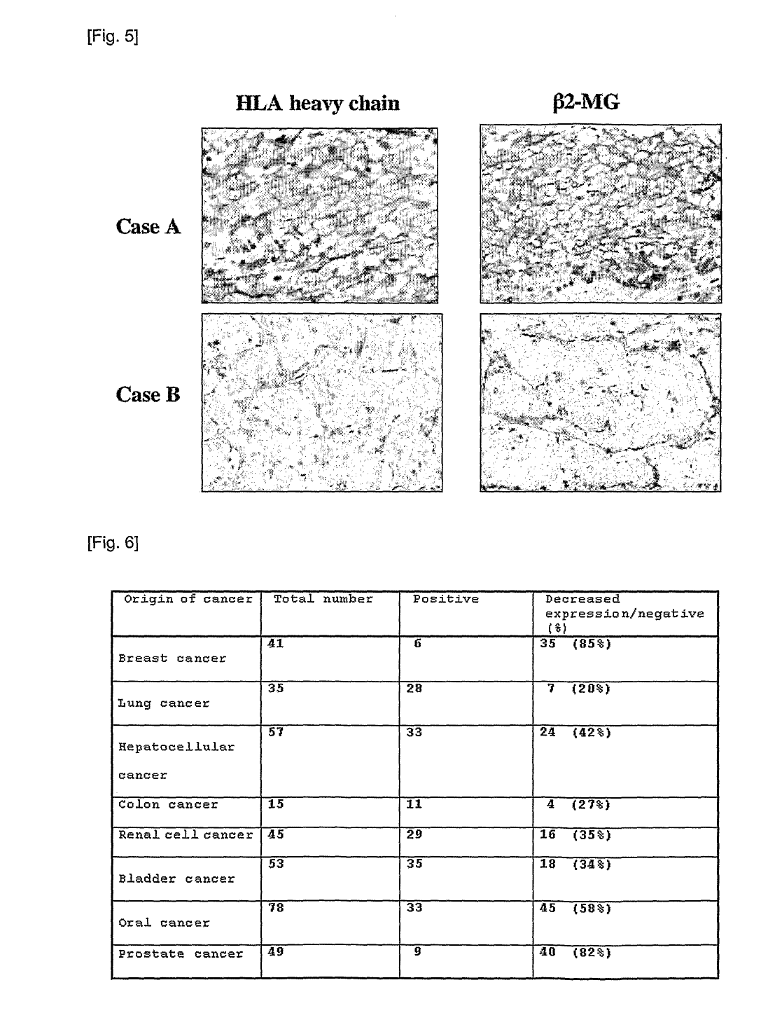

The results showed that there existed the cancer in which HLA class I heavy chain and B2M protein are positive as shown in upper two panels of FIG. 5 (Case A) and the cancer in which HLA class I heavy chain and B2M protein are negative as shown in lower two panels (Case B).

### Mode for the Invention 5

The expression level of MHC class I antigen in various cancer tissues of human was analyzed by a similar immunostaining method to that of example 5. As shown in FIG. 6, the expression of HLA class I antigen was decreased or disappeared in 35 of 41 cases (85%) in breast cancer, and the expression of each HLA class I antigen was decreased or negative in 7 of 35 cases (20%) in lung cancer, 24 of 57 cases (42%) in liver cancer, 4 of 15 cases (27%) in colon cancer, 16 of 45 cases (35%) in renal cell cancer, 18 of 53 cases (34%) in bladder cancer, 45 of 78 cases (58%) in oral cancer and 40 of 49 cases (82%) in prostate cancer. It was noticed that the frequency of decreased expression was remarkably higher in breast cancer and prostate cancer.

### Mode for the Invention 6

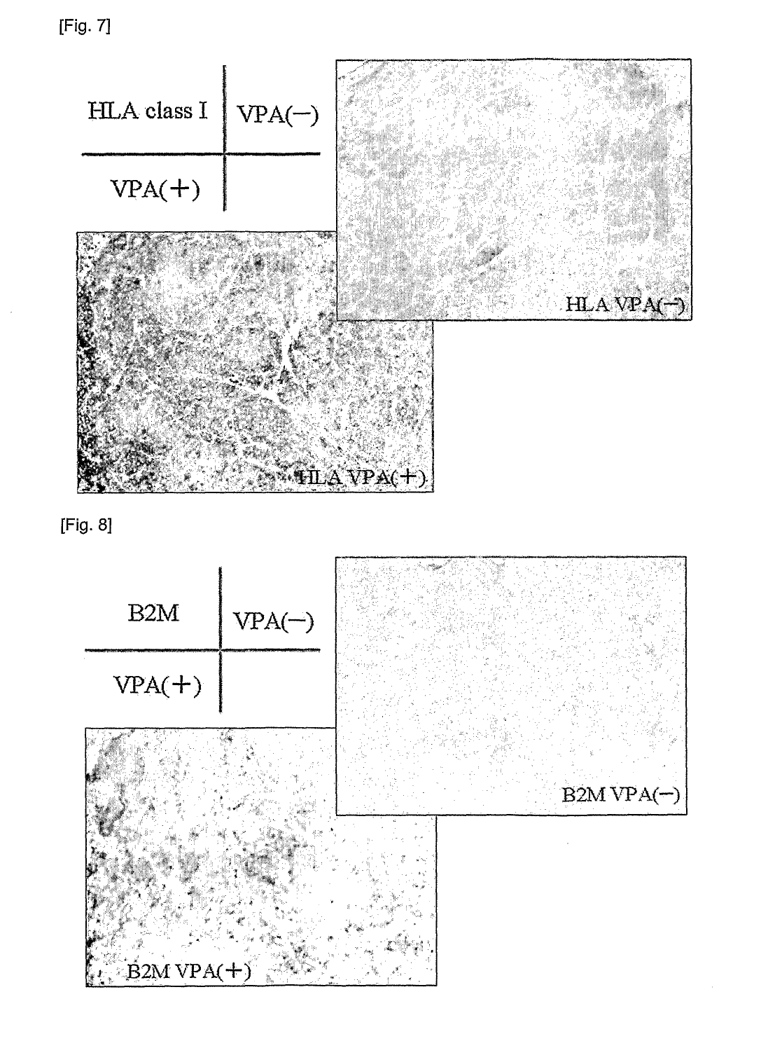

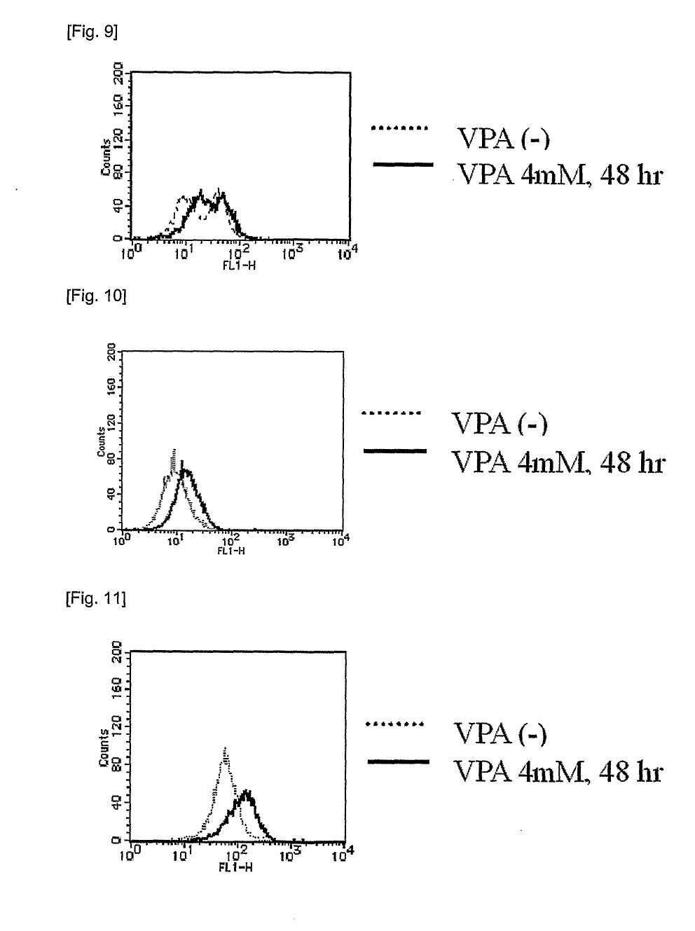

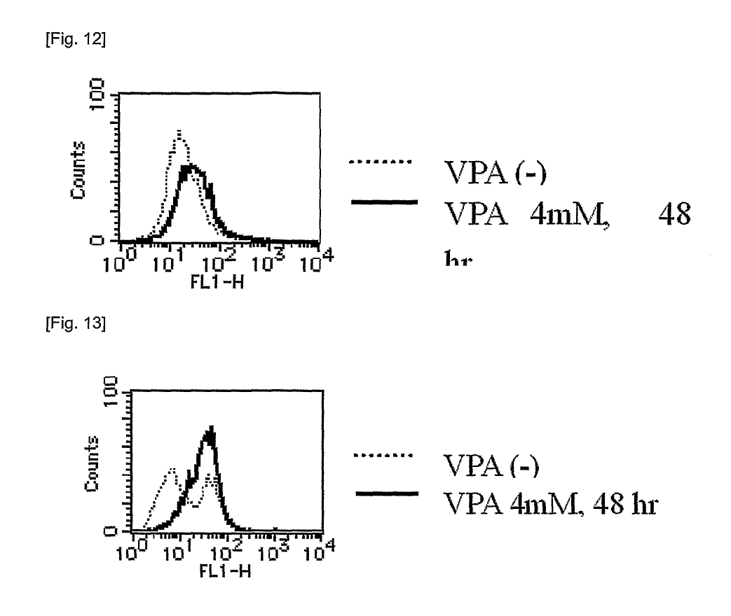

A human breast cancer cell line MCF7 cells were transplanted into SCID mice. After two weeks, subcutaneous tumor of about 5 mm in diameter was obtained. One mouse was supplied with water containing 0.4% valproic acid (VPA, Sigma) for 10 days, and the other mouse was supplied with water with no additive as control. After ten days, the tumor was excised surgically, and 10% formalin (as the mass was small, the concentration was set lower than in example 4 to prevent overfixation) was used to fix overnight at room temperature, and paraffin-embedded slices were obtained. Following the method according to example 4, treatment of tissue slices as well as immunostaining with anti-HLA class I antibody (EMR8-5) and anti-B2M antibody (EMR-B6) were performed. As a result, as shown in FIG. 6 and FIG. 7, the expression levels of HLA class I antigen and B2M were increased compared to control group in the tumor in the VPA-administrated mouse. From this experiment, it was shown to be possible to increase MHC class I antigen expression of human breast cancer tissue by oral administration of an HDAC inhibitor VPA.

### Mode for the Invention 7

Mode of HIV Infection System

HIV infection T cells (clone 8E5, purchased from ATCC) were cultured in 4 mM valproic acid-added culture medium (RPMI1640) or non additive culture medium for 48 hours, and reacted with an anti-pan-HLA class I antibody, W6/32 antibody and with FITC-labeled anti-mouse IgG antibody for 40 minutes respectively, and the cells were fixed in 0.1% formalin/PBS. The fluorescence intensities were measured by flow cytometer (FACScan, Beckton Dickinson). The recovery of expression was observed in the infected cell group in which the HLA class I level on a cell surface was decreased, in the presence of the HDAC inhibitor.

### Mode for the Invention 8

Papilloma virus persistent infection cells (HeLa cells) were cultured in 4 mM valproic acid-added culture medium (RPMI1640) or non additive culture medium for 48 hours, and reacted with an anti-pan-HLA class I antibody, W6/32 antibody and with FITC-labeled anti-mouse IgG antibody for 40 minutes respectively, and the cells were fixed in 0.1% formalin/PBS. The fluorescence intensities were measured by flow cytometer (FACS can, Beckton Dickinson). The recovery of expression was observed in the infected cell group in which the HLA class I level on a cell surface was decreased, in the presence of the HDAC inhibitor.

### Mode for the Invention 9

EB virus persistent infection B cells (LG2EBV cells) were cultured in 4 mM valproic acid-added culture medium (RPMI1640) or non additive culture medium for 48 hours, and reacted with an anti-pan-HLA class I antibody, W6/32 antibody and with FITC-labeled anti-mouse IgG antibody for 40 minutes respectively, and the cells were fixed in 0.1% formalin/PBS. The fluorescence intensities were measured by flow cytometer (FACScan, Beckton Dickinson). The recovery of expression was observed in the infective cell group in which the HLA class I level on a cell surface was decreased, in the presence of the HDAC inhibitor.

### Mode for the Invention 10

Mumps virus persistent infection B cells (Akata-MP1 cells) were cultured in 4 mM valproic acid-added culture medium (RPMI1640) or non additive culture medium for 48 hours, and reacted with an anti-pan-HLA class I antibody, W6/32 antibody and with FITC-labeled anti-mouse IgG antibody for 40 minutes respectively, and the cells were fixed in 0.1% formalin/PBS. The fluorescence intensities were measured by flow cytometer (FACScan, Beckton Dickinson). The recovery of expression was observed in the infective cell group in which the HLA class I level on a cell surface was decreased, in the presence of the HDAC inhibitor.

### Mode for the Invention 11

Measles virus persistent infection B cells (Raji-ZH cells) were cultured in 4 mM valproic acid-added culture medium (RPMI1640) or non additive culture medium for 48 hours, and reacted with an anti-pan-HLA class I antibody, W6/32 antibody and with FITC-labeled anti-mouse IgG antibody for 40 minutes respectively, and the cells were fixed in 0.1% formalin/PBS. The fluorescence intensities were measured by flow cytometer (FACScan, Beckton Dickinson). The recovery of expression was observed in the infective cell group in which the HLA class I level on a cell surface was decreased, in the presence of the HDAC inhibitor.

In all the virus infection systems mentioned above, although any elevation of B2M expression was not observed by application of the HDAC inhibitor, an elevation of MHC class I expression was observed in a similar way. The results were thoroughly unexpected, even compared with the observation in cancer tissues.

1-23. (canceled)

24. An enhancer of cellular immunity comprising a substance having an inhibitory activity of histone deacetylase (HDAC) as an active ingredient.

25. The enhancer of cellular immunity according to claim 24, wherein the substance having an inhibitory activity of histone deacetylase (HDAC) is valproic acid.

26. The enhancer of cellular immunity according to claim 24, used for treatment or prevention of a disease in which β2-microglobulin expression is suppressed at the disease site.

27. The enhancer of cellular immunity according to any one of claims 24 to 26, wherein the disease is an infection caused by a pathogenic microorganism.

28. The enhancer of cellular immunity according to claim 27, wherein the pathogenic microorganism is a virus.

29. The enhancer of cellular immunity according to claim 28, wherein the virus is one or a plurality of viruses selected from the group consisting of members of the herpesvirus, papovavirus, lentivirus and paramyxovirus families.

30. The enhancer of cellular immunity according to any one of claims 24 to 26, wherein the disease is a cancer.

31. The enhancer of cellular immunity according to claim 30, wherein the cancer is a breast cancer.

32. The enhancer of cellular immunity according to claim 30, wherein the cancer is a prostate cancer or an oral cancer.

33. An enhancer of HLA class I expression comprising a substance having an inhibitory activity of histone deacetylase (HDAC) as an active ingredient.

34. An enhancer of β2-microglobulin expression comprising a substance having an inhibitory activity of histone deacetylase (HDAC) as an active ingredient.

35. A method for enhancing cellular immunity comprising a step of dosing a subject a composition containing a substance having an inhibitory activity of histone deacetylase (HDAC) as an active ingredient.

36. The method for enhancing cellular immunity according to claim 35, wherein the substance having an inhibitory activity of histone deacetylase (HDAC) is valproic acid.

37. The method for enhancing cellular immunity according to claim 35, used for treatment or prevention of a disease in which β2-microglobulin expression is suppressed at the disease site.

38. The method for enhancing cellular immunity according to any one of claims 35 to 37, wherein the subject is infected with a pathogenic microorganism.

39. The method for enhancing cellular immunity according to claim 38, wherein the pathogenic microorganism is a virus.

40. The method for enhancing cellular immunity according to claim 39, wherein the virus is one or a plurality of viruses selected from the group consisting of members of the herpesvirus, papovavirus, lentivirus and paramyxovirus families.

41. The method for enhancing cellular immunity according to any one of claims 35 to 37, wherein the disease is a cancer.

42. The method for enhancing cellular immunity according to claim 41, wherein the cancer is a breast cancer.

43. The method for enhancing cellular immunity according to claim 41, wherein the cancer is a prostate cancer or an oral cancer.

44. A method for screening a subject whose cellular immunity is to be enhanced by use of a substance having an inhibitory activity of histone deacetylase (HDAC) comprising:
1) a step of obtaining an infective tissue or cancer tissue from the subject,
2) a step of extracting β2-microglobulin or a mRNA encoding β2-microglobulin from said tissue, and
3) a step of comparing an amount of said β2-microglobulin or said mRNA encoding β2-microglobulin with an amount of a control.

45. A method for screening a subject whose cellular immunity is to be enhanced by use of a substance having an inhibitory activity of histone deacetylase (HDAC) comprising:
1) a step of obtaining an infective tissue or cancer tissue from the subject,
2) a step of performing chromatin immunoprecipitation with said tissues to obtain precipitate, and performing PCR amplification of DNA fragments of β2-microglobulin from said precipitate to obtain amplified DNA fragments, and
3) a step of determining whether said amplified DNA fragments exist.

46. The method according to claim 44 or 45, wherein the substance having an inhibitory activity of histone deacetylase (HDAC) is valproic acid.
